(19) United States
(12) Patent Application Publication
(10) Pub. No.: US 2025/0172538 A1
(43) Pub. Date: May 29, 2025

(54) MAGNETIC ACTUATION SYSTEM FOR TISSUE ENGINEERING

(71) Applicant: The Johns Hopkins University, Baltimore, MD (US)

(72) Inventors: Deok-Ho Kim, Seattle, WA (US); Gwangjun Go, Baltimore, MD (US); Devin Mair, Dundalk, MD (US)

(21) Appl. No.: 18/837,483

(22) PCT Filed: Feb. 10, 2023
(86) PCT No.: PCT/US23/62445

§ 371 Date: Aug. 9, 2024

Related U.S. Application Data
(60) Provisional application No. 63/308,908, filed on Feb. 10, 2022.

(51) Int. Cl. G01N 33/483; A61B 5/243; A61B 5/248; C12M 1/42; C12N 13/00

(52) U.S. Cl. CPC G01N 33/4833; A61B 5/243; A61B 5/248; C12M 35/02; C12N 13/00

(57) ABSTRACT

In one aspect, a magnetic actuation system for tissue engineering is provided. A preferred includes a) a magnetic substrate; and b) a magnetic field source adapted for dynamic application of a magnetic field to the magnetic substrate

### CROSS-REFERENCE TO RELATED APPLICATION

This application claims the benefit of U.S. provisional application 63/308,908 filed Feb. 10, 2022, which is incorporated by reference herein in its entirety.

### STATEMENT REGARDING FEDERALLY SPONSORED RESEARCH

This invention was made with government support under grant UG3TR003271 and UH3TR003271 awarded by the National Institutes of Health. The government has certain rights in the invention.

### FIELD

The present disclosure relates to a magnetic actuation system for tissue engineering.

### BACKGROUND

Engineered tissue has an excellent potential as a 3D microphysiological system to regenerate damaged organs, for more physiological preclinical drug screening, and for disease modeling. So far, these engineered tissues have been focused on forming 2D and 3D models, identifying detailed structures and biological properties and testing them for drug toxicity/screening, but their ability to mimic the physiology of tissues in in vivo environments has been limited. While these in vivo tissues are exposed to various internal and external environments and are constantly stimulated, cells cultured in vitro have limitations in growing and maintaining fully mature and functional tissues in an isolated and established culture environment.

Recently, in order to overcome this limitation, methods of applying external stimuli such as mechanical, electrical, and hydrodynamics to engineered tissue have been studied to mimic thein vivo environment, which can result in tissue properties and structure more mimetic of human physiology. In particular, among them, is the use of mechanical actuation has been done by placing engineered tissue inside a bioreactor system and applying mechanical stress via on a hydraulic device or a magnetic field. As a result, this tissue actuation approach using such a system improved our understanding of the biological properties of tissues and cells. However, most bioreactor systems consist of bulky and complex mechanical parts. Since most of the tissues used have a centimeter scale, there is a limit to uniformly delivering nutrients and drugs to the tissues as a whole and optically observing images of the tissues due to their high thickness. In addition, due to the narrow workspace, the number of tissues that can be subjected to mechanical actuation at one time is small, so the bioreactor systems developed so far has low throughput.

More recently, engineered tissue platforms based on micro/nanofabricated structures with various sizes and shapes have been developed. These structures are manufactured based on microelectromechanical systems (MEMS), micromachining, and replica molding technology. A representative example, a platform with two posts, which is easy to form and can support 3D tissue, is being widely applied. The micro/nanofabricated structure-based platform has a large number of structure arrays, so it has the advantage of being able to support a large number of micro-and submilli-scale tissues on a small chip. Several research groups have proposed a platform based on two posts driven by an external magnetic field to transfer mechanical stress to the tissue formed in the structure array [1-3]. The post of the platform contains a magnetic responsive material, which enables the mechanical stress on the tissue to be adjusted according to the intensity and period of the external magnetic field. Furthermore, it has been shown that the force applied to the tissue by mechanical actuation could be obtained by mathematically calculating the deflection of the post.

### SUMMARY

We have found that in the case of microtissues formed on a plurality of two posts-based platforms are observed through an inverted microscope, mechanical actuation in multiple tissues cannot be observed simultaneously in low throughput. In addition, post-based platforms can provide only the tensile force of tissue using a magnetic field, so there is a limit to simulating various types of forces occurring in tissues in vivo, such as skeletal muscles and the heart.

In one aspect, we now provide a magnetic actuation system for tissue engineering, that includes a magnetic field forming unit for stimulating the tissue.

In preferred aspects the magnetic field can be applied dynamically, i.e. in a real-time on and off state, distinct from use of a permanent magnetic.

In one preferred aspect, the magnetic field forming unit is an electromagnetic coil system.

In a preferred aspect, the system further includes a device for actuation of analyzed biological tissue, such as cells, tissue (e.g. muscle fiber) or engineered tissue, including myocardial or lung tissue. In one embodiment, the actuation device suitably is a post-array stem. In another embodiment, the actuation device can be magnetized materials (e.g. beads or nanoparticles) that can be applied to tissue for analysis.

In one aspect, a magnetic actuation system for biological material analysis is provided, comprising: a) a magnetic substrate; and b) a magnetic field source adapted for dynamic application of a magnetic field to the magnetic substrate.

In another aspect, a magnetic actuation system for biological material analysis is provided, comprising: a) a magnetic substrate; and b) a magnetic field coil system proximate to the magnetic substrate material.

In yet another aspect, a magnetic actuation system for biological material is provided, comprising: a) a magnetic substrate; and b) a magnetic field source that at least partially circumscribes the magnetic substrate material.

The magnetic field forming unit suitably at least partially circumscribes the magnetic substrate material, i.e. is positioned wherein a magnetic field can be applied from multiple planes, e.g. at least 2, 3 or 4 different planes relative to the magnetic substrate material. The magnetic field forming unit also suitably is positioned in a position proximate to the magnetic substrate material whereby an effective magnetic field strength can be applied to the magnetic substrate material.

In one embodiment, the magnetic material suitably comprises a magnetic post array.

In another embodiment, the magnetic substrate comprises magnetic particles. In a particular aspect, the magnetic substrate comprises magnetic nanoparticles.

In an aspect, the system comprises biological material, for example as loaded onto, bound to or proximate to the magnetic substrate. The biological material may comprise for example cells, tissue or engineered tissue, including myocardial or lung tissue, or muscle fiber. In an aspect, the biological material may be contacted or coated with magnetic particles or nanoparticles.

In an aspect, the system may further comprise an optical or magnetic-based monitoring unit for observing biological material condition.

In an aspect, the magnetic substrate comprises a post system that comprises at least one flexible post and one magnetic responsive material for actuating and sensing biological material.

In aspects, the magnetic substrate comprises a material comprising Fe, Co, Mn, Ni, Gd, Mo, MM′₂O₄, MₓO_{y} (M and M′ are each independently Fe, Co, Ni, Mn, Zn, Gd, or Cr, x is an integer of 1 to 3, and y is an integer of 1 to 5), CoCu, CoPt, FePt, CoSm, NiFe, and NiFeCo.

In aspects, the magnetic substrate comprises a post system that comprises a post element that comprises a polymeric or glass material. In aspects, the magnetic substrate comprises a post system that comprises a post element that comprises PDMS, Ecoflex, and/or Dragon Skin.

In aspects, the system comprises an electromagnetic coil for applying an electric field to the magnetic substrate.

In aspects, the tension and torsion of biological material by attractive and repulsive forces of the post are manipulated by controlling the magnetic field forming unit.

We have found that tissue including engineered tissue formed on a post array device was stretched by the actuation of the post magnetized by the magnetic field. The behavior of the tissues could be observed by a monitoring unit during the magnetic actuation simultaneously.

According to an aspect of the present invention, provided is a magnetic actuation system for tissue engineering, comprising: multiple posts array for formation and actuation of tissue; an optical or magnetic-based monitoring unit for observing tissue condition; and a magnetic field forming unit for stimulating the tissue.

In another aspect, a method is provided for stimulating tissue or other biological material such as cells by magnetic actuation. In particular aspects, the tissue or other biological material such as cells are stimulated by magnetic actuation and without any mechanical actuation or other mechanical manipulation of the biological material during the analysis step.

The present disclosure relates to a magnetic actuation system for cell and tissue (biological material) engineering therefor. In one aspect, the magnetic actuation system of the present disclosure comprises 1) multiple posts array, and 2) a magnetic field forming unit. The magnetic actuation system can provide various types of magnetic field-based actuation to the tissue formed between the posts.

The system suitably may further comprise an optical or magnetic-based monitoring unit to monitor the biological tissue during agentic field actuation.

In addition, the system can act as a medium-and high-throughput system by applying the same magnetic field to a plurality of posts arrays.

Further, magnetic and optical sensing units allow observation of biological material (tissue) conditions during magnetic actuation of the tissue.

The present systems and methods are particularly useful for assessment, diagnosis and/or treatment of a variety of diseases and disorders.

In particular, the present systems and methods are useful for assessment, diagnosis and/or treatment of a myocardial tissue or a disease or disorder relating to myocardial tissue.

The present systems and methods are also useful for assessment, diagnosis and/or treatment of a lung tissue or a disease or disorder relating to lung tissue, or assessment, diagnosis or treatment involving of muscle fiber.

The present systems, chips and methods are particularly useful for assessment, diagnosis and/or treatment of muscular dystrophies, including Duchenne Muscular Dystrophy, XL-MTM, Nemaline myopathy, myotonic dystrophy.

The present systems, chips and methods are particularly useful for assessment, diagnosis and/or treatment of cardiomyopathies, including hypertrophic cardiomyopathy (HCM), dilated cardiomyopathy (DCM), Heart failure with preserved ejection fraction (HFpEF), Pompe disease, and arrhythmogenic cardiomyopathy (ACM).

The present systems, chips and methods are particularly useful for assessment, diagnosis and/or treatment of peripheral neuropathies, including Charcot-Marie-Tooth (CMT), Amyotrophic Lateral Sclerosis (ALS).

The present systems and methods could be utilized to study Frank-Starling mechanism in dystrophic or healthy tissues.

Engineered tissue has excellent potential as a 3D microphysiological system to regenerate damaged organs, for more physiological preclinical drug screening, and for disease modeling.

The present systems and methods have been shown to improve tissue function compared to tissues cultured under no mechanical actuation. The present systems suitably can be coupled with e.g. electrical pacing or biochemical methods to further improve maturation and function of engineered tissues, particularly those derived from pluripotent stem cells.

The present systems and methods can assess mechanical stretch and load in variety of materials and tissue to assess pathological outcomes. Examples of mechanical loading and stretch in pathological circumstances include: 1) Mechanical stretch is found on cardiac fibroblasts following myocardial infarction in the infarct zone. Stretch is also applied to the cardiomyocytes in the border region surrounding the infarct zone. These mechanical forces result in myofibroblast activation via mechanical signaling and activation of downstream biochemical pathways. 2) Effects of mechanical loading on muscle injury. 3) Stretch-induced lung injury due to ventilation. 4) Study of natural cyclic mechanical stretch in the lung and the effects on cellular processes. 5) study of hydronephrosis in the kidney. 6) Bladder stretch injury. 7) Study of systolic aortic stretch. 8) Study of the effects of mechanical load and stretch in cancer migration and development. 9) Study of the effects of low magnetic fields in proliferation and differentiation of cell differentiation. 10) Study of magnetically actuated micro/nanocomposites for targeted delivery of therapeutic agents. 11) Magnetic stimulation of mechanosensitive ion channels for mechanobiological studies. Other pathologies across numerous tissue types are affected by mechanical loading, which can be further studied using the system described.

In one particular system, we evaluated the use of actuation in terms of myocardial infarction-induced mechanical stretch for inducing cardiac dysfunction and potential fibrosis. Magnetic actuation at higher forces in the system described herein were indeed found to cause attenuation of tissue function, indicative of pathological effects (100 μN condition). Similarly, 100 μN forces resulted in significantly higher relative stiffness, indicating potential fibrosis, as fibrotic tissue is known to have higher stiffness. Drug screening can be performed in this system to help identify efficacious therapeutics for mechanical loading and stretch induced pathological insults.

Further uses of the present actuation systems include applying forces directly to single cells such as through magnetic nanoparticles. Such systems with nanoparticles suitably may be used for purposes such as cell sorting, spatial guidance into separate cell niches to mimic natural cell organization, or study mechanics of the cell membrane, among others. Additionally, such systems may be utilized to guide magnetic nanomedicines in vivo in animal models or in engineered tissues to test efficacy in a specific organ type or spatial area.

In one specifically preferred system, a magnetic actuation system comprises 1) a magnetic substrate that comprises one or more multiple posts arrays, 2) an optical or magnetic-based monitoring unit, and 3) a magnetic field forming unit as disclosed herein, e.g. a magnetic field source adapted for dynamic application of a magnetic field to the magnetic substrate and/or a magnetic field coil system proximate (i.e. a distance effective to provide an analysis or actuation effective magnetic field) to the magnetic substrate material.

The magnetic actuation system can provide various types of magnetic field-based actuation to the tissue formed between the posts. Furthermore, it can act as a medium-and high-throughput system by applying the same magnetic field to a plurality of posts arrays.

Other aspects of the invention are disclosed infra.

### BRIEF DESCRIPTION OF THE DRAWINGS

FIGS. 1A is a schematic illustration of a magnetic field forming unit in accordance with the present disclosure.

FIG. 1B is a schematic illustration of multiple posts array containing engineered tissue and a monitoring unit for observing tissue actuation in accordance with the present disclosure.

FIG. 2A is a schematic illustration of electromagnetic coils as a magnetic field forming unit in accordance with the present disclosure.

FIG. 2B shows simulation results of magnetic fields generated from each coil in accordance with the present disclosure.

FIG. 3A is an image of fabricated electromagnetic coils as a magnetic field forming unit in accordance with the present disclosure.

FIG. 3B shows measurement results of magnetic fields within the workspace of electromagnetic coils in accordance with the present disclosure.

FIG. 4A shows an experimental setup of a magnetic field forming unit and a monitoring unit in accordance with the present disclosure.

FIG. 4B shows an optical camera-mounted motorized stages in accordance with the present disclosure.

FIG. 5 shows an image of two posts on a 24-well plate, including silicone rubber for mimicking engineered tissue in accordance with the present disclosure.

FIG. 6 indicates the working principle and parameters of two posts using magnetic field control in accordance with the present disclosure.

FIG. 7A shows snapshots of magnetic actuation of silicone rubber on two sets of post array according to intensity of the magnetic fields in accordance with the present disclosure.

FIG. 7B shows snapshots of magnetic actuation of silicone rubber on two sets of post array according to the direction of the magnetic fields in accordance with the present disclosure.

FIG. 8A is a schematic illustration of two magnets-embedded posts containing engineered tissue in accordance with the present disclosure.

FIG. 8B is a schematic illustration of twisting and tilting motions induced by magnetic actuation of two magnets-embedded posts in accordance with the present disclosure.

FIG. 9 shows snapshots of magnetic actuation of silicone rubber on two magnets-embedded posts according to the direction of the magnetic fields in accordance with the present disclosure.

FIG. 10 (includes FIGS. 10A-10B) shows magnetic actuation can model positive functional effects of mechanical loading and stretch. FIG. 10A: Engineered heart tissues were actuated at the specified forces for 3 weeks. FIG. 10B: Actuation results in stepwise increase in relaxation velocity at lower forces.

FIG. 11 (includes FIGS. 11A-11C) shows magnetic actuation can model negative pathological effects of mechanical loading and stretch. Engineered heart tissues were actuated at the specified forces for 3 weeks. FIG. 11A: Engineered heart tissues actuated at 100 μN have significantly attenuated force production. FIG. 11B: Actuation at 100 μN results in significant decreases in relaxation velocity. FIG. 11C: Actuation at higher forces results in insignificant decreases but modest decreases in contraction velocity.

FIG. 12 shows 100 μN actuation forces result in higher relative tissue stiffness as measured by difference in tissue length under 100 μN forces. Each tissue was recorded three times. There is no significant difference

FIG. 13 (includes FIGS. 13A-13C) shows actuation forces represented as percent of tissue twitch forces, tissue displacements, and percent strain. (A) Actuation forces chosen range between ˜20% and ˜80 of mean tissue twitch forces. (B) Actuation forces chosen result in displacements of ˜50 μm to ˜200 μm. (C) Actuation forces chosen result in ˜0.5 to ˜2.5% strain on the tissues.

FIGS. 14A-14D shows week by week fold change measurements of contractile kinetics across all actuation forces. Measurements are taken as a fold change versus the previous week, with week 0 being the baseline recording. (A) Twitch force, (B) frequency, (C) contraction velocity, and (D) relaxation velocity fold changes approach one at week 3, indicating no changes between week 2 and week 3.

FIG. 15 depicts a preferred post system with tissue.

FIG. 16 depicts a further multiple post array where magnetic fields are applied, magnetic field forming unit and monitoring unit.

FIG. 17 depicts mold units for fabrication of polymer post array for present devices.

FIG. 18 depicts additional preferred post array system including with tissue or pseudo-tissue.

FIG. 19 shows arrays of the present systems, including where can be fitted with post arrays and within a support frame.

FIG. 20 further shows array systems including with tissue casting and steps of incubation following placement of tissue in a post system.

FIG. 21 depicts a preferred system that includes an electromagnetic coil system in addition to magnetic actuation system (e.g. post array).

FIG. 22 depicts a preferred system that includes magnetic actuation system (e.g. post array) together with control parameters.

FIG. 23 shows a further preferred magnetic actuation system.

### DETAILED DESCRIPTION

In one aspect, a multiple posts array system is provided for formation and actuation of tissue (biological material). In one system, the composition of the post array includes at least one flexible post and one magnetic responsive material for actuating and monitoring tissue.

In one preferred aspect, the post array involves a flexible post with the magnetic responsive material coupled and a rigid post with a biocompatible material with high strength. A couple of posts are mentioned, but this is a non-limiting example. For example, more than a pair of posts may be included, for example, even or odd posts. In addition, magnetic responsive material and biocompatible material with high strength can be coupled into more than one post, respectively.

In the above composition, the magnetic responsive material may be, but are not limited to, at least one selected from the group consisting of at least one selected from the group consisting of Fe, Co, Mn, Ni, Gd, Mo, MM′2O4, MxOy (M and M′ are each independently Fe, Co, Ni, Mn, Zn, Gd, or Cr, x is an integer of 1 to 3, and y is an integer of 1 to 5), CoCu, CoPt, FePt, CoSm, NiFe, and NiFeCo.

In the above composition, biocompatible material with high strength may be for example poly (lactic-co-glycolic acid) (PLGA), poly (glycolic acid) (PGA), poly (lactic acid) (PLA), polycaprolactone (PCL), Teflon®, PET, and glass and others.

In the above composition, the post material may be, but are not limited to, at least one selected from among others polydimethylsiloxane (PDMS), Ecoflex, and Dragon Skin, as well as other polymers including acrylics.

The post array may be prepared by various methods, for example, micromachining, molding, and 3D printing. In an embodiment, the post array was prepared by the molding method.

The post array suitably may be configured or adjusted to have a post diameter of 1-2 mm, and a post height of 8.5-12.5 mm. These dimensions can be inserted and used in commercial 24- and 96-well plates, or other systems.

In certain systems, the post array may contain tissue aligned to the post as a mixture of cells and extracellular matrix (ECM) is introduced and the cells and mixture compact.

Exemplary tissue to use in the present systems include but is not limited to myocardial, lung, bone, cartilage, bladder, and skeletal muscle tissue types.

According to another aspect, provided is a magnetic actuation system for tissue engineering, which includes: multiple posts array for formation and actuation of tissue; an optical or magnetic-based monitoring unit for observing tissue condition; and a magnetic field forming unit for stimulating the tissue.

In the magnetic actuation system for tissue engineering, the monitoring unit for observing tissue conditions may be a magnetic sensor or an optical camera. As an example, an optical camera is mounted on the motorized stages, but this is a non-limiting example.

In the magnetic actuation system for tissue engineering, the magnetic field forming unit may be coil-type or permanent magnet-type, and the magnetic field formation of the magnetic field forming unit maybe by a soft magnet, permanent magnet, or electromagnet. In particular embodiments, the permanent magnet may be ferrite, neodymium, alnico samarium cobalt, or rubber magnet.

As an example of the magnetic actuation system, the magnetic field forming unit includes one or multiple pairs (e.g. 2, 3, 4 or more) pairs of electromagnetic coils positioned suitably in orthogonal directions and a frame to support them. It can preferably generate a uniform magnetic field to deliver a mechanical force of desired strength to multiple post arrays. Although three pairs of electromagnetic coils are shown, this is a non-limiting example. For example, suitably 1 or up to 8 or more coils can be arranged to apply a uniform magnetic field to multiple post arrays. A post of an array suitably includes a magnetic composition such as neodymium or other magnet incorporated into the post.

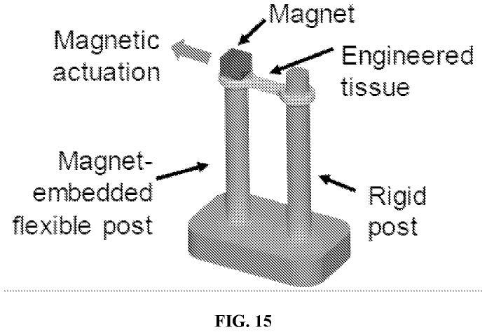

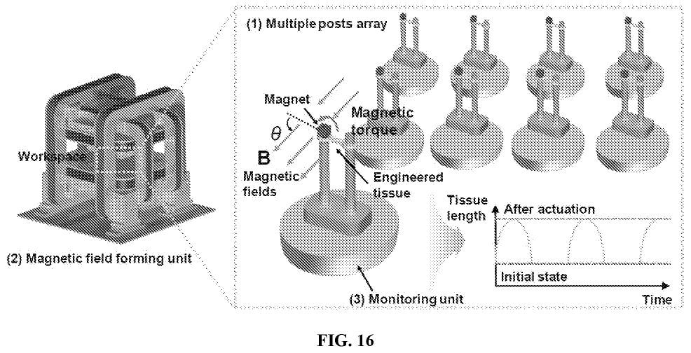

FIG. 15 shows a preferred magnetic actuation system that includes a multiple (dual) post system with a magnet containing post and a second post with tissue therebetween. Direction of magnetic actuation is shown. Such a system can be used to magnetically detect forces in analyzed tissue, including myocardial forces. The depict posts may be fabricated from a variety of materials including various polymers (e.g. an acrylic), glass, and other materials. Additional posts may be employed in a particular array, and post systems may be utilized in parallel or serially.

FIG. 16 shows additional preferred posts (shown in (1), a magnetic field forming unit (shown in (2), and a base monitoring unit (shown in (3)).

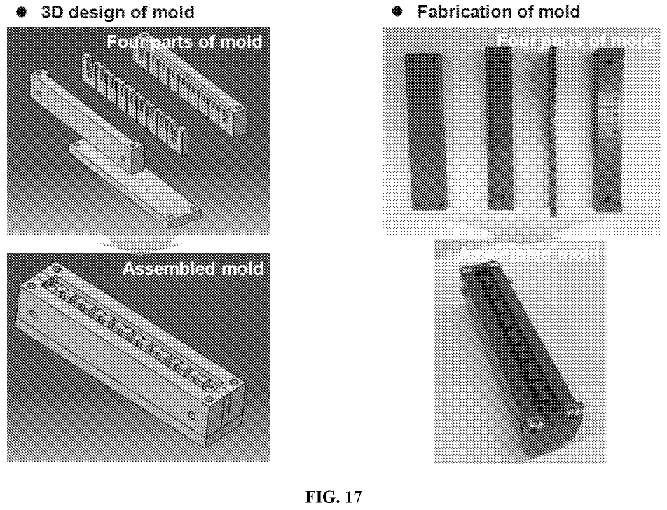

The post arrays such as depicted in FIGS. 15 and 16 may be suitably fabricated by a variety of methods. FIG. 17 depicts a preferred mold for fabricating post arrays where polymeric post arrays may be produced. The molds may be produced for example by 3-D printing.

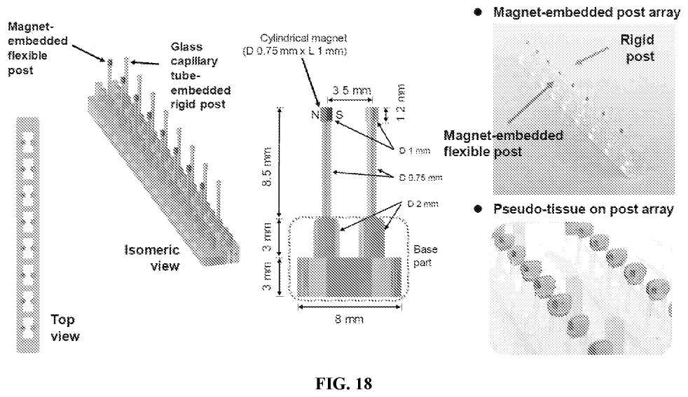

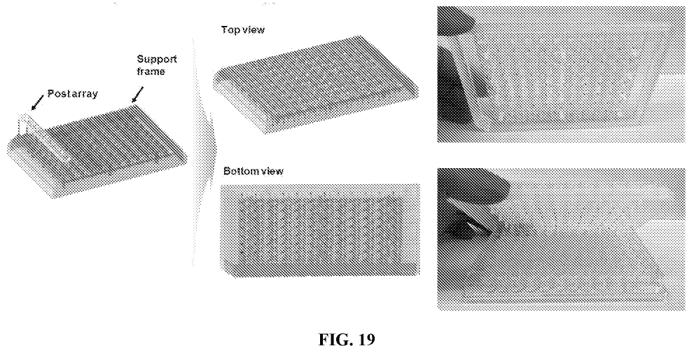

FIG. 18 shows further preferred post systems including positioned in arrays and suitable dimensions and spacing of posts and other elements. FIG. 19 shows preferred high-throughput systems including plate systems that have in excess of 24, 48, 60, 72, 80 or 90 (such as 96 or greater) wells or receptacles for a detection array.

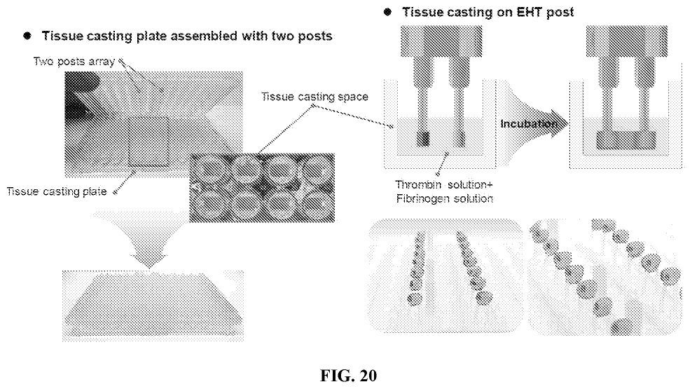

FIG. 20 depicts systems and process for applying or depositing tissue for analysis into a magnetic detection system. As shown tissue may be suitably cast using a plate or other application device onto a post and then the deposited material may be incubated as desired.

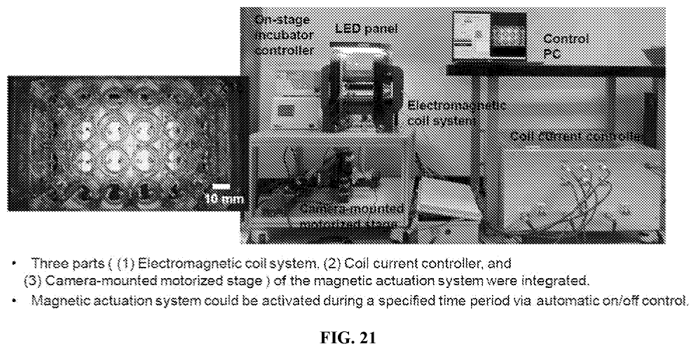

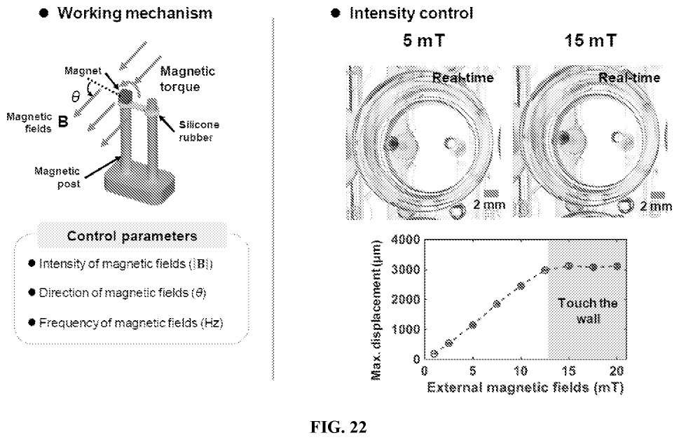

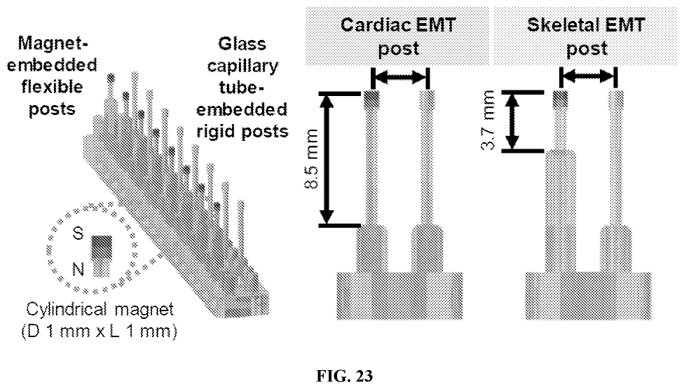

FIG. 21 depicts a preferred system that includes an electromagnetic coil system in addition to magnetic actuation system (e.g. post array). FIG. 22 depicts a preferred system that includes magnetic actuation system (e.g. post array) together with control parameters.

The present invention examples are provided to allow those skilled in the art to more fully understand the present invention, and the following examples may be modified into various other forms. The scope of the present invention is not limited to the following Examples. Rather, these examples are provided so that the present disclosure will be more faithful and complete and will fully convey the scope of the invention to those skilled in the art. Additionally, in the drawings, the thickness and size of each layer are exaggerated for convenience and clarity of explanation.

The examples of the present invention are provided to allow those skilled in the art to more fully understand the present invention, and the following examples may be modified into various other forms, and the scope of the present invention is not limited to the following Examples. Rather, these examples are provided so that the present disclosure will be more faithful and complete, and will fully convey the scope of the invention to those skilled in the art. Additionally, in the drawings, the thickness and size of each layer are exaggerated for convenience and clarity of explanation.

Throughout the specification, when one constituting element (e.g., membrane, area, substrate, etc.) is referred to be located as being “on”, “connected”, “stacked”, or “coupled” to another constituting element, it may be interpreted that the one constituting element is directly in contact with the another constitution element by being “on”, “connected”, “stacked”, or “coupled”, or still other constituting elements to be interposed there between may be present. In contrast, when one constituting element is referred to be located as being “directly on”, “directly connected”, or “directly coupled” to another constituting element, it is interpreted that no other constituting elements to be interposed there between are present. The same reference numeral indicates the same element. As used herein, the term “and/or” includes any one of the listed items and one or more combinations of the listed items.

In the present specification, the terms first, second, etc. are used to describe various elements, components, regions, layers and/or parts, but it is apparent that the elements, components, regions, layers and/or parts should not be limited to these terms. These terms are used only to distinguish one element, component, region, layer or part from another region, layer, or part. Accordingly, a first element, component, region, layer, or part described below may refer to a second element, component, region, layer, or part without departing from the teachings of the present invention.

Additionally, relative terms such as “above” or “over” and “below” or “under” may be used herein to describe the relationship of certain elements to other elements as illustrated in the figures. It may be understood that relative terms are intended to encompass different orientations of the device in addition to the orientation depicted in the figures. For example, in the drawings, when a device is turned over the elements illustrated as being on the upper surface of other elements will have orientations on the lower surface of the other elements. Therefore, the term “above” exemplified may encompass both orientations of “above” and “below” depending on a particular direction in the figures. If an element is directed to a different orientation (rotated 90 degrees with respect to the other orientation), the relative descriptions used herein can be interpreted accordingly.

The terms used herein are for the purpose of describing particular examples only and are not intended to limit the scope of the invention. As used herein, a singular form may include a plural form unless the context clearly indicates otherwise. Additionally, when used in the present invention, “comprise” and/or “comprising” specify the presence of stated features, integers, steps, operations, members, elements, and/or groups thereof, but do not preclude the presence or addition of one or more other features, integers, operations, members, elements, and/or groups.

Hereinafter, examples of the present invention will be described with reference to the drawings schematically showing ideal embodiments of the present invention. In the figures, for example, variations in the shape illustrated may be expected, depending on the manufacturing technique and/or tolerance. Accordingly, the examples of the present invention should not be construed as being limited to the specific shapes of the regions illustrated herein, but should include, for example, changes in shape resulting from manufacture.

An approach has been demonstrated that enables mechanical actuation of engineered tissue by applying a magnetic field to a post array containing a magnetically responsive material to one or more posts.

This approach allows the engineered tissue to mature to resemble that in vivo and transmit tensile and torsional forces to the tissue depending on the number of posts containing the external magnetic field and magnetic responsive material and the orientation of the magnetic responsive material.

Using an external magnetic field generated from the magnetic field forming unit can provide mechanical stimulation to tissue non-invasively compared to an electromagnet having a pole tip shape, which is an invasive probing method inserted into a culture medium.

Furthermore, the force applied to the tissue by magnetic actuation can be measured and analyzed in real-time.

This disclosure provides a device for actuating and maturing engineered tissue particularly those derived from pluripotent stem cells. A multiple post array, a magnetic field forming unit and a monitoring unit works by delivering a uniform force to multiple tissues simultaneously and measuring the extent to which multiple tissues are stretched during mechanical stimulation.

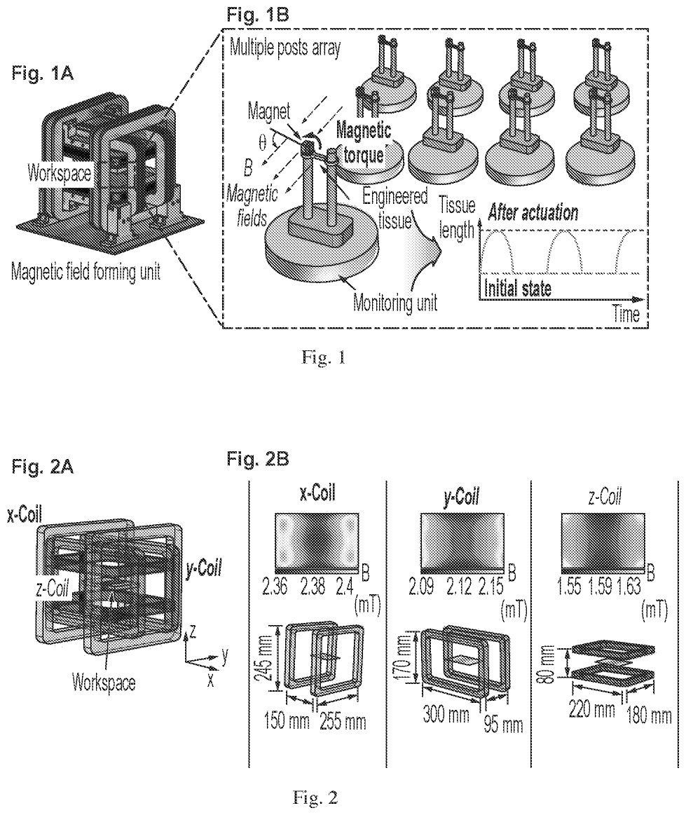

FIG. 1 illustrates a process of actuating of engineered tissue using a magnetic actuation system. FIG. 1(A) shows a magnetic field forming unit that can generate a uniform magnetic field to transmit a desired strength of mechanical force to multiple post arrays, and consists of three pairs of electromagnetic coils positioned in orthogonal directions and a frame for supporting them. However, this is a non-limiting example. FIG. 1(b) illustrates the present approach in an array format, with a schematic of multiple post array and a monitoring unit. One post array includes a flexible post with magnet coupled and a rigid post with glass tube. A pair of posts is shown, but this is a non-limiting example. A mixture of cells and extracellular matrix (ECM) is introduced into the post array, and as the cells and mixture compact, aligned tissue is formed between the posts. The magnetic field generated from the magnetic field forming unit in an arbitrary direction (θ) generates a magnetic torque in the magnet-embedded flexible post, and the tissue is stretched as the flexible post is bent. Here, the tissue stretching by magnetic field-based mechanical actuation is observed and recorded in real time via a monitoring unit.

FIG. 1 illustrates a process of actuating engineered tissue using a magnetic actuation system. FIG. 1(A) shows a magnetic field forming unit that can generate a uniform magnetic field to transmit the desired strength of the mechanical force to multiple post arrays and consists of three pairs of electromagnetic coils positioned in orthogonal directions and a frame for supporting them. However, this is a non-limiting example. FIG. 1(b) illustrates the current approach in an array format, with a schematic of multiple post arrays and a monitoring unit. One post array includes a flexible post with magnet coupled and a rigid post with a glass tube. A pair of posts are shown, but this is a non-limiting example. A mixture of cells and extracellular matrix (ECM) is introduced into the post array, and as the cells and mixture compact, aligned tissue is formed between the posts. The magnetic field generated from the magnetic field forming unit in an arbitrary direction (θ) generates a magnetic torque in the magnet-embedded flexible post, and the tissue is stretched as the flexible post is bent. Here, the tissue stretching by magnetic field-based mechanical actuation is observed and recorded in real-time via a monitoring unit.

FIG. 2 shows the design and simulation results for the magnetic field forming unit. FIG. 2A illustrates the electromagnetic coils with three pairs of coils arranged orthogonally. The workspace of electromagnetic coils is 130 mm×90 mm×30 mm (Width×Length×Height) and can contain multiple post arrays combined with commercial 24- and 96-well plates. FIG. 2B shows simulation results of magnetic fields generated from each coil. The magnetic field map in the workspace according to a current of 1A applied to each coil was obtained through COMSOL software. As a result, it was confirmed that the magnetic field in the workspace generated from each coil had an error of within 5%. Therefore, this result indicates that the magnetic field forming unit can provide the same mechanical actuation to all tissues by applying a uniform magnetic field to multiple post arrays located on the well plate.

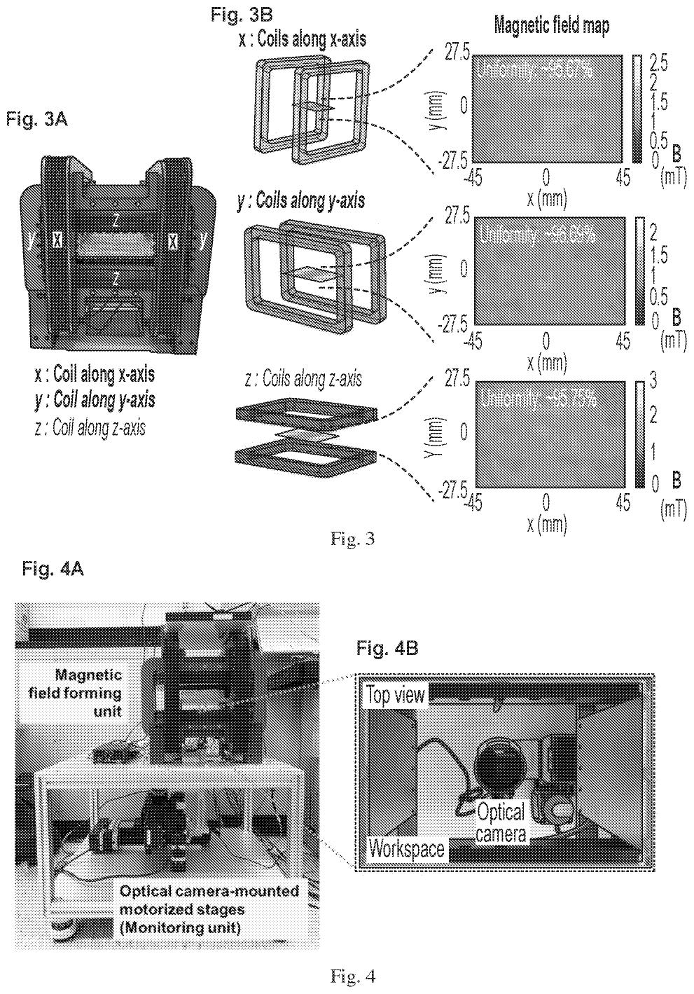

FIG. 3 shows the fabricated magnetic field forming unit and the magnetic field characterization result generated therefrom. FIG. 3A shows an image of a magnetic field forming unit consisting of three pairs of electromagnetic coils and a frame to support them. To confirm the characterization related to the magnetic field generation of the manufactured magnetic field forming unit, the magnetic field generated from each coil by applying a current of 1A was measured using a magnetic sensor in the workspace. As shown in FIG. 3B, the magnetic field generated in the workspace from each coil shows the uniformity of more than 95%, and this result indicates that it has a similar tendency to the simulation result (FIG. 2B). Thus, the magnetic actuation system can give almost the same force to the multiple posts array.

FIG. 4 shows an image of a monitoring unit combined with a magnetic field forming unit. As shown in FIG. 4A, the monitoring unit consists of an optical camera and coupled motorized stages. The optical camera can be moved under the control of the motorized stages, and thus the entire workspace inside the magnetic field forming unit can be observed (FIG. 4B).

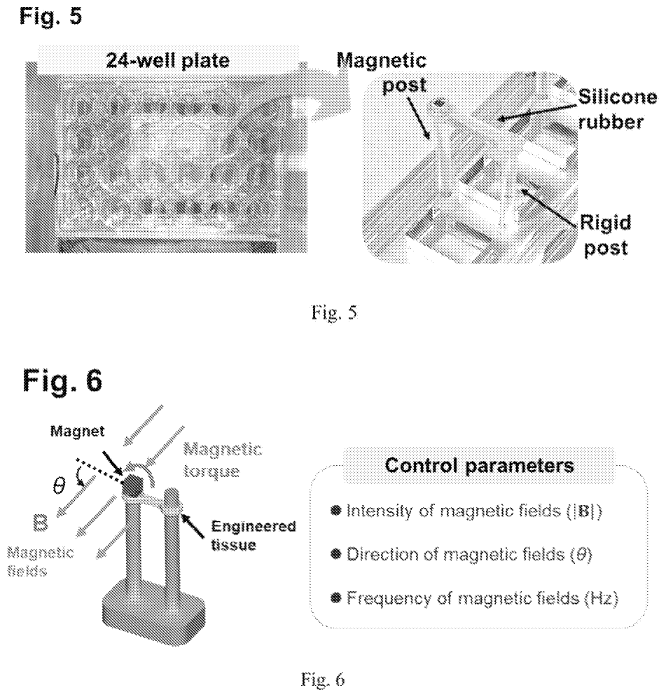

As a preliminary test of mechanical actuation on the tissue using the magnetic field forming unit, magnetic actuation of post on a 24-well plate was observed. Two sets of the post array were placed inside the workspace of the magnetic actuator (FIG. 5). Each post set consists of one magnet-embedded flexible post, one rigid post, and muscular tissue-mimicking silicone rubber.

In one example embodiment of the multiple post array, the posts were constructed using PDMS in a custom mold. First, a small amount of PDMS was poured into the mold and baked at 65° C. to form the caps at the tips of the posts. After forming the caps, 1 mm³ cubic magnets were placed into sections of the mold that formed the flexible posts. Glass capillary tubes (1.1 mm diameter) were placed into sections of the mold that formed the rigid posts. PDMS was then poured into the entire mold and cured overnight at 65° C. External magnets were used to hold the embedded magnets in place and maintain their orientation at the bottom of the posts while the PDMS was cured. After the posts were removed from the mold, excess PDMS was trimmed away, yielding silicone posts that were 12.5 mm tall (including the 0.5 mm tall caps), 1.5 mm diameter, and spaced 8 mm apart (FIG. 5). Tissue-mimicking silicone rubber was obtained through the replica molding method. Specifically, an appropriate amount of Ecoflex 00-20 was poured into the prepared mold and cured at room temperature for 4 hours. Then, the tissue-mimicking silicone rubber was carefully separated from the mold.

After the setup of the hardware, the working mechanism for mechanical actuation was determined. As shown in FIG. 6, when the magnetic field forming unit generates the magnetic fields to the direction (θ), the flexible post is deflected to the direction (θ) by the magnetic torque of the magnet. Resultantly, tissue-mimicking silicone rubber is stretched by the deflection of the magnetic post. The flexible post's deflection and periodical motion can be adjusted by controlling three parameters (i.e., intensity, direction, and frequency of the magnetic fields).

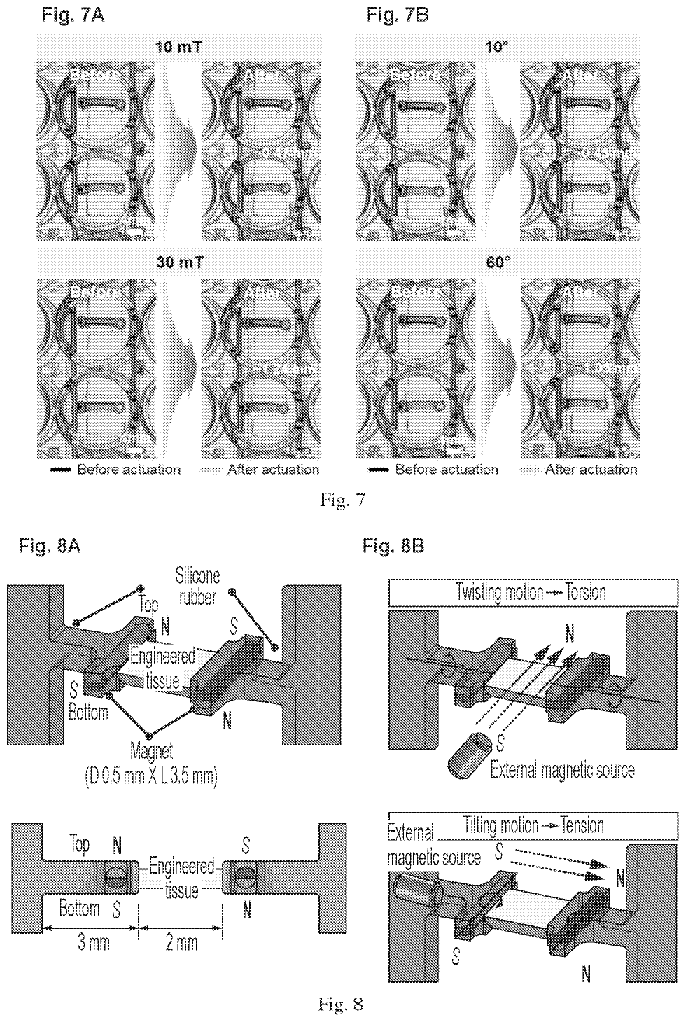

Based on this experimental setup, preliminary tests were performed on the mechanical actuation of the post array. To verify the controllability of bending and periodical motion, its motion was tested according to changing intensity, direction, and frequency of the magnetic fields. As an experimental method, the intensity of the magnetic field was adjusted from 10 mT to 30 mT, and its direction was maintained at 90° (FIG. 7A). The direction of magnetic fields was changed from 10° to 60°, and its intensity was maintained at 30 mT (FIG. 7B). As a result, we confirmed that the magnetic post's deflection increases with the intensity and direction of magnetic fields. These results show the feasibility of the proposed magnetic actuation system that artificial mobility, including strength and rate, of engineered tissue can be imparted by using magnetic field-based mechanical actuation.

As an example of another type of post array, two magnets-embedded flexible posts are designed for magnetic actuation of engineered tissue. FIG. 8 shows a schematic illustration of two magnets-embedded flexible posts that explain the detailed structure and working mechanism of the post array. As shown in FIG. 8A, the magnet attached to the two posts are each magnetized in opposite directions, and these magnets are driven in different directions by the direction of the external magnetic fields. However, this is a non-limiting example. The number of posts, as well as magnets, can be adjusted. A mixture of cells and ECM is introduced into the post array, and as the cells and mixture compact, aligned tissue is formed between the posts. FIG. 8B shows the possible motions using two magnets-embedded flexible posts. The two posts can implement twisting and tilting motions according to the direction of the external magnetic field, which in turn can transmit torsional and tensile forces to the tissue between the posts, respectively (top and bottom of FIG. 8B, respectively).

In one example embodiment of the multiple post array, the posts were constructed using Ecoflex 00-20 in a custom mold. First, two cylindrical magnets (Diameter 0.5 mm×Length 3.5 mm) were placed into sections of the mold that formed the flexible posts. After then, a small amount of Ecoflex 00-20 was poured into the mold and cured at room temperature. External magnets were used to hold the embedded magnets and maintain their orientation at the bottom of the posts while the Ecoflex 00-20 was cured. After the posts were removed from the mold, excess Ecoflex 00-20 was trimmed away, yielding silicone posts that were 3 mm side length, 0.8 mm² cross-sectioned area, and spaced 2 mm apart (FIG. 8A). Tissue-mimicking silicone rubber was obtained through the replica molding method.

Specifically, an appropriate amount of Ecoflex 00-20 was poured into the prepared mold and cured at room temperature for 4 hours. Then, the tissue-mimicking silicone rubber was carefully separated from the mold.

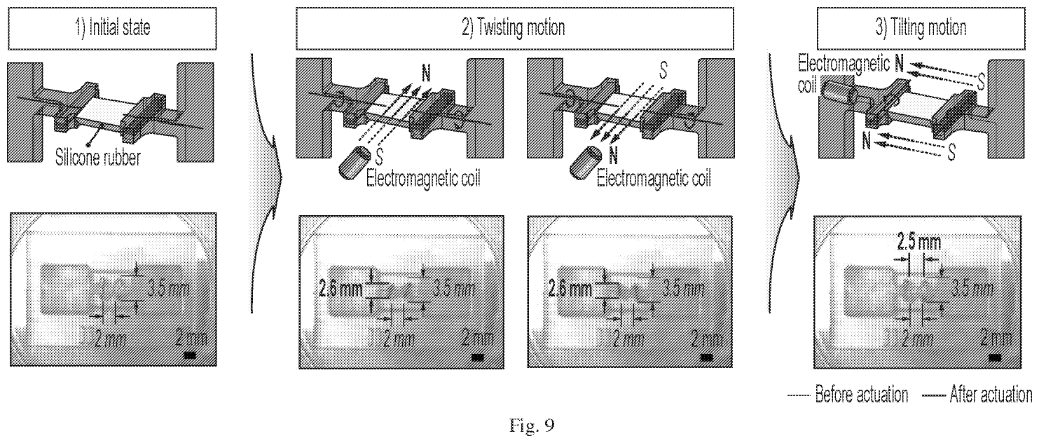

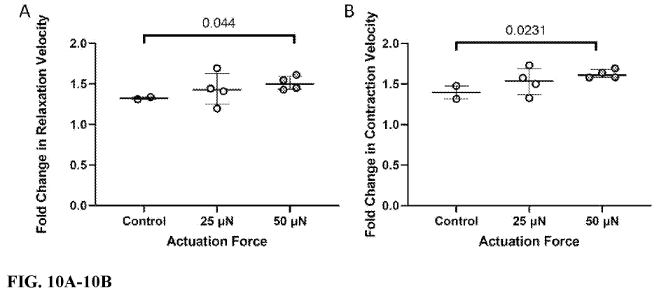

As a preliminary test of mechanical actuation of the engineered tissue using the magnetic field forming unit, magnetic actuation of two posts on a 24-well plate was observed. As shown in FIG. 9, The two magnets-embedded posts could implement different motions such as twisting and tilting by changing the direction of an external magnetic field with a constant intensity of 30 mT. These motions induced the shape change of the tissue-mimicking silicone, and the twisting and tilting motions caused a twist angle of 65 and a length change of 500 μm compared to the initial state, respectively. Consequently, the two-magnets embedded posts can mimic in vivo cardiac motion by delivering torsional and tensile forces to tissue-mimicking silicone.

Also, for further suitably systems, see Xu et al., Lab Chip, 2015, 15, 2496; and Javor et al., Journal of Microelectronic Systems, vol. 30, No. 1, pages 96-104, February 2021, and U.S. Pat. No. 11,331,027 all of which are incorporated herein in their entirety.

The following non-limiting Example is illustrative.

### Example 1

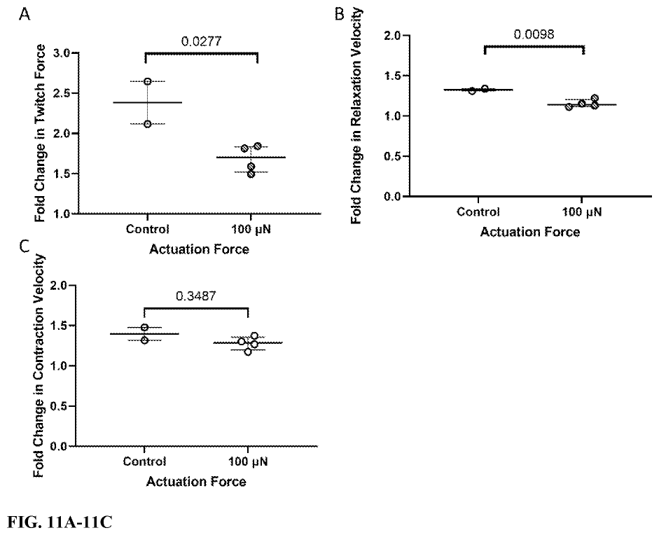

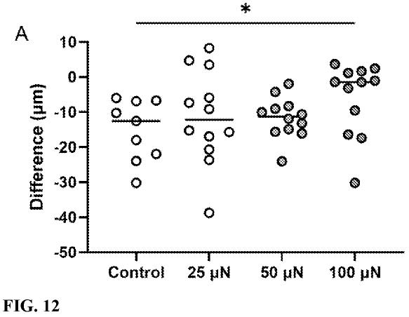

We specifically investigated the use of actuation in terms of myocardial infarction-induced mechanical stretch for inducing cardiac dysfunction and potential fibrosis. Magnetic actuation at higher forces in the system described herein were indeed found to cause attenuation of tissue function, indicative of pathological effects (FIG. 11, 100 μN condition). Similarly, 10 0μN forces resulted in significantly higher relative stiffness (FIG. 12), indicating potential fibrosis, as fibrotic tissue is known to have higher stiffness (15). Drug screening can be performed in this system to help identify efficacious therapeutics for mechanical loading and stretch induced pathological insults.

Another use for this actuation system includes applying forces directly to single cells via magnetic nanoparticles with minimized adverse effects for purposes such as cell sorting, spatial guidance into separate cell niches to mimic natural cell organization, or to study mechanics of the cell membrane, among other uses (16). It can similarly be utilized to guide magnetic nanomedicines in vivo in animal models or in engineered tissues to test efficacy in a specific organ type or spatial area (17).

### Methods

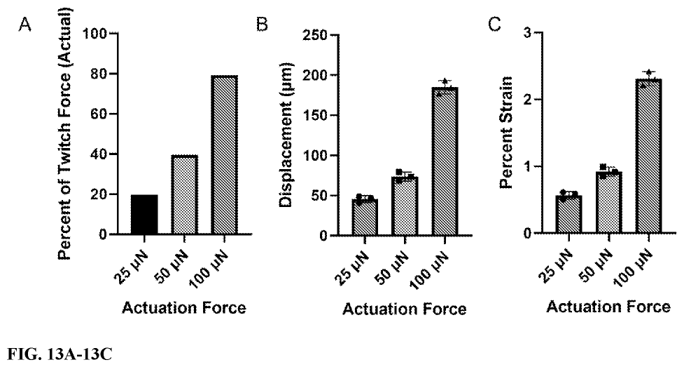

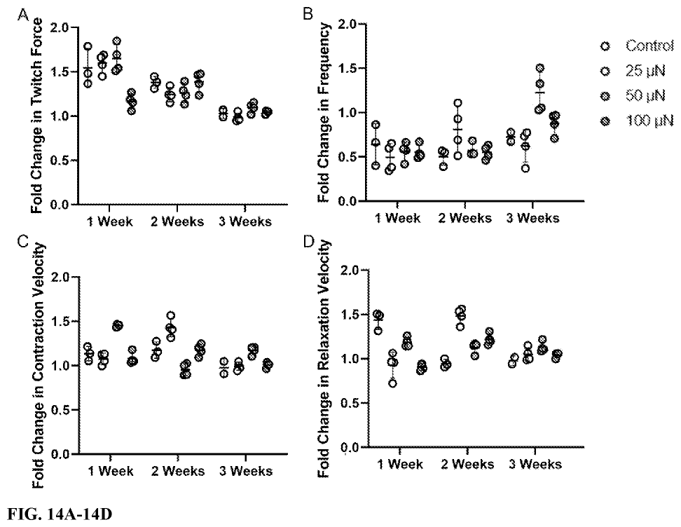

Engineered heart tissues were cast as previously described (18). A baseline recording was taken at day five. One week following this recording, tissues were magnetically actuated at 25, 50, or 100 μN. These actuation forces resulted in a range of about 20% to 80% of tissue twitch forces, displacements of the tissue of 50 to 200 microns, and percent strains of about 0.5% to 2.5% (FIG. 13). Control tissues were kept in the same incubator but were not actuated. Tissue contractile kinetics were recorded weekly using the commercially available Mantarray system. Week by week fold change measurements, taken as the fold change from the previous week, were compared and the experiment was terminated at week 3 of actuation as the fold change of most metrics approached one (meaning no change) across all conditions at this time (FIG. 14). Fold change measurements in FIG. 1 and FIG. 2 were taken as week 3 measurements normalized to their baseline recording on week 1 to compare development of tissue function. Relative stiffness measurements were taken by recording tissues under 100 μN magnetic forces and measuring the difference in tissue length, similar to a tensile test, where higher values indicate a higher relative stiffness.

### REFERENCES

1. Huebsch N, Charrez B, Neiman G, Siemons B, Boggess S C, Wall S, et al. Metabolically driven maturation of human-induced-pluripotent-stem-cell-derived cardiac microtissues on microfluidic chips. Nat Biomed Eng. 2022 April; 6(4):372-88.
2. Ronaldson-Bouchard K, Ma S P, Yeager K, Chen T, Song L, Sirabella D, et al. Advanced maturation of human cardiac tissue grown from pluripotent stem cells. Nature. 2018 April; 556(7700):239-43.
3. van Putten S, Shafieyan Y, Hinz B. Mechanical control of cardiac myofibroblasts. J Mol Cell Cardiol. 2016 April; 93:133-42.
4. Camelliti P, Gallagher J O, Kohl P, McCulloch A D. Micropatterned cell cultures on elastic membranes as an in vitro model of myocardium. Nat Protoc. 2006; 1(3):1379-91.
5. Herum K M, Choppe J, Kumar A, Engler A J, McCulloch A D. Mechanical regulation of cardiac fibroblast profibrotic phenotypes. Mol Biol Cell. 2017 Jul. 7; 28(14):1871-82.
6. Loerakker S, Stekelenburg A, Strijkers G J, Rijpkema J J M, Baaijens F P T, Bader D L, et al. Temporal effects of mechanical loading on deformation-induced damage in skeletal muscle tissue. Ann Biomed Eng. 2010 August; 38(8):2577-87.
7. Mitsui Y, Koutsogiannaki S, Fujiogi M, Yuki K. In Vitro Model of Stretch-Induced Lung Injury to Study Different Lung Ventilation Regimens and the Role of Sedatives. Transl Perioper Pain Med. 2020; 7(3):258-64.
8. López-Martínez C, Huidobro C, Albaiceta G M, López-Alonso I. Mechanical stretch modulates cell migration in the lungs. Ann Transl Med. 2018 January; 6(2):28.
9. Thotakura R, Anjum F. Hydronephrosis And Hydroureter. In: StatPearls [Internet]. Treasure Island(FL):StatPearls Publishing; 2022 [cited 2023 Feb. 6]. Available from: http://www.ncbi.nlm.nih.gov/books/NBK563217/10.
10. Halachmi S. The molecular pathways behind bladder stretch injury. J Pediatr Urol. 2009 February; 5(1):13-6.
11. Plonek T, Rylski B, Nawrocki P, Beyersdorf F, Jasinski M, Kuliczkowski W. Systolic stretching of the ascending aorta. Arch Med Sci AMS. 2019 Feb. 18; 17(1):25-30.
12. Gong F, Yang Y, Wen L, Wang C, Li J, Dai J. An Overview of the Role of Mechanical Stretching in the Progression of Lung Cancer. Front Cell Dev Biol [Internet]. 2021 [cited 2023 Feb. 6]; 9. Available from: https://www.frontiersin.org/articles/10.3389/fcell.2021.781828
13. Berrueta L, Bergholz J, Munoz D, Muskaj I, Badger G J, Shukla A, et al. Stretching Reduces Tumor Growth in a Mouse Breast Cancer Model. Sci Rep. 2018 May 18; 8(1):7864.
14. Liu Q, Luo Q, Ju Y, Song G. Role of the mechanical microenvironment in cancer development and progression. Cancer Biol Med. 2020 May 15; 17(2):282-92.
15. Yoo A, Go G, Nguyen K T, Lee K, Min H K, Kang B, et al. Magnetoresponsive stem cell spheroid-based cartilage recovery platform utilizing electromagnetic fields. Sens Actuators B Chem. 2020 Mar. 15; 307:127569.
16. Go G, Yoo A, Nguyen K T, Nan M, Darmawan B A, Zheng S, et al. Multifunctional microrobot with real-time visualization and magnetic resonance imaging for chemoembolization therapy of liver cancer. Sci Adv. 2022 Nov. 18; 8(46):eabq8545.
17. Lee J U, Shin W, Lim Y, Kim J, Kim W R, Kim H, et al. Non-contact long-range magnetic stimulation of mechanosensitive ion channels in freely moving animals. Nat Mater. 2021 July; 20(7):1029-36.
18. Conrad C H, Brooks W W, Hayes J A, Sen S, Robinson K G, Bing O H L. Myocardial Fibrosis and Stiffness With Hypertrophy and Heart Failure in the Spontaneously Hypertensive Rat. Circulation. 1995 January; 91(1):161-70.
19. Yaman S, Anil-Inevi M, Ozcivici E, Tekin H C. Magnetic Force-Based Microfluidic Techniques for Cellular and Tissue Bioengineering. Front Bioeng Biotechnol. 2018 Dec. 19; 6:192.
20. Estelrich J, Escribano E, Queralt J, Busquets M A. Iron Oxide Nanoparticles for Magnetically-Guided and Magnetically-Responsive Drug Delivery. Int J Mol Sci. 2015 Apr. 10; 16(4):8070-101.
21. Mair D B, Williams M A C, Chen J F, Goldstein A, Wu A, Lee P H U, et al. PDMS-PEG Block Copolymer and Pretreatment for Arresting Drug Absorption in Microphysiological Devices. ACS Appl Mater Interfaces. 2022 Aug. 31; 14(34):38541-9.

While the present invention has been described with reference to the foregoing examples, it is apparent to those skilled in the art that these examples are only for illustrative purposes and various modifications and equivalent embodiments are possible without departing from the scope of the present invention. Accordingly, the true scope of the present invention should be determined by the technical idea of the appended claims.

1. A magnetic actuation system for biological material analysis, comprising:
a) a magnetic substrate; and
b) a magnetic field source adapted for dynamic application of a magnetic field to the magnetic substrate.

2. A magnetic actuation system for biological material analysis, comprising:
a) a magnetic substrate; and
b) a magnetic field coil system at a distance to provide an analysis or actuation effective magnetic field to the magnetic substrate material.

3. A magnetic actuation system for biological analysis, comprising:
a) a magnetic substrate; and
b) a magnetic field source that at least partially circumscribes the magnetic substrate material.

4. The system of claim 1 wherein the magnetic material comprises a magnetic post array.

5. The system of claim 1 wherein the magnetic substrate comprises magnetic particles.

6. (canceled)

7. The system of any one of claims 1 through 6 further comprising biological material.

8-9. (canceled)

10. The system of claim 7 wherein the biological material is muscle fiber.

11. The system of claim 7 wherein the biological material is contacted with magnetic particles or nanoparticles.

12. (canceled)

13. The system of claim 1 further comprising an optical or magnetic-based monitoring unit for observing biological material condition.

14. The system of claim 1 wherein the magnetic substrate comprises a post system that comprises at least one flexible post and one magnetic responsive material for actuating and sensing biological material.

15-17. (canceled)

18. The system of claim 1 comprises an electromagnet for applying an electric field to the magnetic substrate.

19. (canceled)

20. A method for treating or stimulating biological material by magnetic actuation, comprising:
a) loading a system of claim 1 with biological material; and
b) applying a dynamic magnetic field to the biological material.

21. A method for treating or stimulating biological material by magnetic actuation, comprising magnetic actuation system for biological material analysis, comprising:
a) loading a system of claim 1 with biological material; and
b) applying a magnetic field to the magnetic substrate material with a magnetic field coil system.

22. (canceled)

23. A method for treating or stimulating biological material by magnetic actuation, comprising:
a) loading a system of claim 1 with biological material; and
b) applying a magnetic field to the magnetic substrate material with a magnetic field source that at least partially circumscribes the magnetic substrate material.

24-29. (canceled)

30. A system or method of claim 1 used for assessment, diagnosis and/or treatment of muscular dystrophies, including Duchenne Muscular Dystrophy, XL-MTM, Nemaline myopathy, and myotonic dystrophy.

31. A system or method of claim 1 used for assessment, diagnosis and/or treatment of cardiomyopathies, including hypertrophic cardiomyopathy (HCM), dilated cardiomyopathy (DCM), Heart failure with preserved ejection fraction (HFpEF), Pompe disease, and arrhythmogenic cardiomyopathy (ACM).

32. A system or method of claim 1 used for assessment, diagnosis and/or treatment of peripheral neuropathies, including Charcot-Marie-Tooth (CMT) and Amyotrophic Lateral Sclerosis (ALS).

33. A system or method of claim 1 used for assessment, diagnosis and/or treatment of a myocardial tissue or a disease or disorder relating to myocardial tissue.

34. A system or method of claim 1 used for assessment, diagnosis and/or treatment of a lung tissue or a disease or disorder relating to lung tissue.

35. A system or method of claim 1 used for assessment, diagnosis and/or treatment of muscle fiber.
